(19) United States
(12) Patent Application Publication
(10) Pub. No.: US 2010/0015068 A1
(43) Pub. Date: Jan. 21, 2010

(54) Methods and Compositions For Altering Biological Surfaces

(75) Inventors: Jeffrey M. Karp, BROOKLINE, MA (US); Alireza Khademhosseini, Cambridge, MA (US); David A. Berry, Brookline, MA (US); Robert S. Langer, Newton, MA (US); Frank X. Gu, Cambridge, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/309,049

(22) PCT Filed: Jul. 6, 2007
(86) PCT No.: PCT/US07/15525

§ 371 Date: Sep. 11, 2009

Related U.S. Application Data
(60) Provisional application No. 60/818,509, filed on Jul. 6, 2006.

(51) Int. Cl. A61K 8/24; C01B 25/32; B32B 5/16; A61K 33/42; A61Q 11/00; A61P 19/10

(52) U.S. Cl. 424/57; 423/308; 428/402; 424/602

(57) ABSTRACT

Compositions, methods and kits are provided for altering the properties of a biological surface using particles such as, for instance, calcium based particles in combination with an agent that binds to the biological surface. Properties such as color, sheen, texture, strength, and odor of biological surfaces such as teeth and bone are alterable.

### BACKGROUND OF INVENTION

Teeth are composed of an inner dentin layer and a hard outer enamel layer. The enamel layer, composed on the hardest material in the body, functions to protect the teeth and provide strength. The enamel layer is composed of a translucent calcium phosphate mineral, hydroxyapatite, which forms microscopic hexagonal rods. Light passes through the tooth enamel and is reflected by the dentin, creating the normal “pearly white” appearance of teeth. Exposure to certain foods, tobacco, or coffee/tea can stimulate the formation of colored pellicle over the enamel layer, which can be typically removed through brushing with abrasive toothpaste or chemical treatments. Unless the pellicle layer is thoroughly removed on a consistent basis, long term exposure can cause foreign material to be incorporated into the enamel, therefore leading to discoloration. The stained appearance specifically results from pores between the hydroxyapatite crystals becoming filled with foreign colored material. In addition to exposure related stains, teeth may also be naturally discolored.

Tooth whitening has rapidly developed into a robust business. Dentist-dispensed whitening products grossed $2 billion in 2005, up from $435 million in 2000—a 35% compound annual growth rate-according to the Mintel International group, a market research firm. A wide range of products are offered for teeth whitening with variable costs, efficacy, and requirements for trained personnel for their use. Home treatments, including paint-on or white-strip whitening agents typically cost −$20. Dentists will make custom plastic trays for use at home with a bleaching gel. The trays typically cost $500-600, and the bleach runs $70-100 per whitening course. This method, however, has notable drawbacks in that during the four consecutive days in which the bleach is to be applied, the teeth become more porous and can be stained much easier. Furthermore, the teeth become significantly more sensitive. Dentists can also make porcelain coatings for teeth, running between $100 and $200 per tooth. Finally, dentists can apply a light-activated gel, which costs upwards of $1000.

Typical tooth whiteners contain bleaching chemicals such as hydrogen peroxide and/or carbamide peroxide that may quickly diffuse into the enamel and dentin (within 15 minutes) where they may begin to oxidize and break apart the staining compounds. Carbamide peroxide breaks down into hydrogen peroxide and urea, with hydrogen peroxide being the active whitening ingredient (10 percent carbamide peroxide is equivalent to 3.6 percent hydrogen peroxide). Most dentist applied systems use 15% to 35% percent peroxide gels, sometimes coupled with a high intensity light to accelerate the bleaching process. Prior to treatment, the dentist gently cleans the teeth with pumice followed by application of a protective coating on the gums. The treatment typically involves of hydrogen peroxide paste or gel application and rinsing. Although this procedure generally achieves four to six shades of whitening after one 40-minute treatment, it may cost upwards of $1000.

Over the counter systems typically use 10% to 20% carbamide peroxide gels containing glycerin, carbomer, sodium hydroxide, water, and flavoring agents. Gels that contain over 10% carbamide peroxide may also include sodium fluoride to reduce sensitivity and strengthen teeth. Although at-home procedures supervised by dentists are generally effective, these procedures typically cost between $300-$500 and require the dentist to take impressions (molds) to fabricate soft custom mouth trays (nightguards) which takes time and thus delays a desired outcome. To administer the treatment, the patient puts a thin layer of the gel into the tray and wears it for two hours during the day, or while sleeping. Most whitening occurs in one to two weeks while in difficult cases, trays may be needed for up to six weeks.

The most popular over the counter products use either carbamide or hydrogen peroxide gels or hydrogen peroxide containing polyethylene strips. Although these products are available for immediate use and typically cost between $10-35, it may take several weeks for a desired outcome and many of the products do not list the concentration of the whitening agents or contain alternatives of varying strengths. Also, systems that use trays or strips may not adequately cover the teeth, and less than desired results or irritation to the gums could occur. Hydrogen peroxide can increase temperature sensitivity in the teeth, particularly at higher concentrations, and nightguards often cause gum irritation. And overzealous use of over-the counter home bleaching products can wear away tooth enamel, especially with solutions that contain acid.

All of the teeth whitening techniques used today have notable drawbacks. The inexpensive over-the-counter products take one-to-six weeks to produce a desired outcome and are typically, not as effective as more expensive techniques. The more effective techniques are substantially more costly with significant potential side effects for cosmetic enhancement. Recently studies, for example, have demonstrated that bleaching may cause damage to teeth, increase tooth sensitivity, stimulate gingival irritation, and negatively influence dental restorations and restoration materials. Furthermore, whitening products today are not universally available. Patients with decayed teeth, infected gums, white spots on their teeth, and multiple tooth colored fillings or crowns (caps) on the front teeth may not be good candidates for tooth whitening. Safer, more robust, and faster acting strategies are therefore in great demand. The ability to produce a rapid, safe, and inexpensive yet effective whitening agent for at-home or dentist office use would be a breakthrough in this market, with the potential to rapidly take a dominating position.

Hydroxyapatite, the major component of teeth, is also a major component of bone. Bone is a living tissue that is constantly undergoing remodeling. When the balance between breakdown and rebuilding is disturbed—for example, by hormonal changes or dietary changes—the bone may lose some of the minerals that contribute to its density and strength. A condition of diminished bone density is called osteopenia. When a significant loss in bone density occurs, such that the bone is markedly weakened and susceptible to fracture, the condition is termed osteoporosis. According to the National Osteoporosis Foundation (NOF), 10 million people in the United States have osteoporosis and another 34 million have low bone mass and are at risk of developing the disease. Of those who have osteoporosis, 80% are women. Together, osteoporosis and osteopenia are expected to affect an estimated 52 million women and men age 50 and older by 2010, and 61 million by 2020. Direct medical costs of osteoporosis total nearly $18 billion in the U.S. each year.

### SUMMARY OF INVENTION

Aspects of the invention relate to methods and compositions for targeting particles to specific regions of the body such as to a tooth or bone. Methods and compositions herein that relate to delivery of a particle to a tooth have applications that include tooth whitening and drug delivery to the oral cavity. Methods and compositions herein that relate to delivery of a particle to bone have applications that include treatment of osteoporosis and other bone disorders.

Aspects of the invention relate to compositions comprising particles. In some embodiments, the composition comprises a calcium particle linked to a hydroxyapatite binding agent. In some embodiments the calcium particle comprises a calcium salt such as calcium carbonate, calcium phosphate, calcium citrate, calcium nalate, calcium gluconate, calcium silicate or calcium stearate. In some embodiments, the hydroxyapatite binding agent comprises bisphosphonate.

Aspects of the invention relate to particles. In some embodiments the particle is a nanoparticle and in other embodiments the particle is a microparticle. In some embodiments the diameter of the calcium particle is about 5 nm to about 500 nm. In other embodiments the diameter of the calcium particle is about 100 nm to about 200 nm. In other embodiments the diameter of the calcium particle is about 10 nm to about 100 nm.

The particle may comprise an aggregate of particles. In some embodiments the aggregate of particles is homogeneous, while in other embodiments the aggregate of particles is heterogeneous. In some embodiments the diameter of the aggregate of particles is less than about 500 nm. In other embodiments the diameter of the aggregate of particles is about 500 nm to about 10 μm. In other embodiments the diameter of the aggregate of particles is about 10 μm to about 100 μm.

Aspects of the invention relate to the binding of a particle to a tooth. In some embodiments the particle is linked to a hydroxyapatite binding agent that binds to hydroxyapatite on the surface of a tooth, binding the particle to the tooth. In some embodiments the composition containing the particle comprises a liquid formulation. In certain embodiments the composition comprises a dentifrice including a mouthwash, a mouthrinse, a toothpaste, a tooth powder, a tooth hardener, an antiplaque composition, a dental cream, a dental floss, a liquid, a gel, a chewing gum, including a center-filled gum, a confectionary, including mints, lozenges.

The composition containing the particle may further comprise dental porcelain, dental amalgam, gypsum plaster impression material, gutta percha root canal filling, vulcanite, silicate cement, zinc phosphate cement, cobalt chromium base alloy, acrylic resin, alginate impression material, bis-GMA and composite resins, polysulphide impression material, zinc polycarboxylate cement, glass ionomer cement, dentine resin adhesive, amalgam, galloy, ceramic based cement, or bone cement.

In some embodiments the composition containing the particle may further comprise a biological agent. In certain embodiments the composition may comprise a flavoring agent, an antibacterial, a bleaching agent, a sweetening agent, an antioxidant, a saliva stimulating agent, a breath freshening agent, an antiplaque agent, an anti-inflammatory, an H2 antagonist, a desensitizing agent, a nutrient, a biomolecule, an opacifying agent, an additional pigment, a fluoride ion source, an analgesic, a surfectant or a humectant.

In some embodiments the calcium particle is further linked to a modifying ligand and a hydroxyapatite binding agent. In some embodiments the hydroxyapatite binding agent may bind to hydroxyapatite on the surface of a bone, binding the particle to the bone. In some embodiments the modifying ligand comprises a stealth ligand such as poly(ethylene glycol), hyaluronic acid, dextran, chitosin, or poly(ethylene oxide).

In some embodiments the particle is degradable. In some embodiments the particle is incorporated into a liposome. In other embodiments the composition comprises a solution of particles linked to a hydroxyapatite binding agent wherein the particle is not incorporated into a liposome. In some embodiments the particle linked to the hydroxyapatite binding agent binds to hydroxyapatite on the surface of a tooth.

In some embodiments the particle comprises calcium, zirconium, zinc, magnesium or titanium salt. In certain embodiments the composition comprises a dentifrice including a mouthwash, a mouthrinse, a toothpaste, a tooth powder, a tooth hardener, an antiplaque composition, a dental cream, a dental floss, a liquid, a gel, a chewing gum, including a center-filled gum, a confectionary, including mints, lozenges. In some embodiments the composition further comprising poly(lactic-co-glycolic acid) (PLGA). In some embodiments the composition further comprises a biological agent.

Aspects of the invention relate to methods for binding a particle to a tooth. In some embodiments the method comprises contacting a tooth of a subject with a composition comprising a calcium particle linked to a hydroxyapatite binding agent, to bind the calcium particle to the tooth. In some embodiments the binding of the particle to the tooth promotes whitening of the tooth. In certain embodiments the tooth surface is brushed to remove the protein. layer prior to contacting the tooth with a particle. In some embodiments the steps are repeated to apply multiple layers of the particle to the biological surface. In certain embodiments the tooth whitening is temporary. In some embodiments the method further comprises a method for treating peridontal disease and/or gingivitis.

In some embodiments the method comprises contacting a tooth of a subject with a particle linked to a hydroxyapatite binding agent, wherein the particle is not incorporated into a liposome, to bind the particle to the tooth. In some embodiments the binding of the particle to the tooth promotes whitening of the tooth. In certain embodiments the tooth surface is brushed to remove the protein layer prior to contacting the tooth with a particle. In some embodiments the steps are repeated to apply multiple layers of the particle to the biological surface. In some embodiments the tooth whitening is temporary. In some embodiments the method further comprises a method for treating peridontal disease and/or gingivitis.

In some embodiments of the invention the method comprises (a) applying to the tooth of a subject a coating agent, and (b) contacting the coating agent with a composition comprising a calcium particle linked to a binding agent wherein the binding agent binds to the coating agent on the surface of the tooth to bind the particle to the tooth. In some embodiments the binding of the particle to the tooth promotes whitening of the tooth. In certain embodiments the tooth surface is brushed to remove the protein layer prior to contacting the tooth with a particle. In some embodiments the steps are repeated to apply multiple layers of the particle to the biological surface. In some embodiments the tooth whitening is temporary. In some embodiments the method further comprises a method for treating peridontal disease and/or gingivitis. In certain embodiments the coating agent removes the protein layer on the teeth. In some embodiments the coating agent comprises dilute aqueous acid. In some embodiments the method further comprises a bridging agent that binds to the binding agent and to the surface of the tooth and/or the coating agent.

In some embodiments of the invention the method comprises contacting an oral cavity of a subject with a calcium particle linked to a saliva or bacterial binding agent to bind the calcium particle to saliva or bacteria on a tooth surface. In some embodiments the binding of the particle to the tooth promotes whitening of the tooth. In certain embodiments the tooth surface is brushed to remove the protein layer prior to contacting the tooth with a particle. In some embodiments the steps are repeated to apply multiple layers of the particle to the biological surface. In some embodiments the tooth whitening is temporary. In some embodiments the method further comprises a method for treating peridontal disease and/or gingivitis.

Aspects of the invention relate to kits for applying a particle to a tooth. In some embodiments a kit comprises a container housing a calcium particle linked to a binding agent, and instructions for applying the calcium particle linked to the binding agent to a tooth. In some embodiments the kit further comprises a container housing a coating agent. In certain embodiments the coating agent is applied to the teeth prior to the application of the particle. In some embodiments the particle binds to the coating agent. In certain embodiments the binding agent is a hydroxyapatite binding agent. In some embodiments the kit further comprises an applicator. In some embodiments the kit further comprises an adhesive. In some embodiments the kit comprises a dentifrice including a mouthwash, a mouthrinse, a toothpaste, a tooth powder, a tooth hardener, an antiplaque composition, a dental cream, a dental floss, a liquid, a gel, a chewing gum, including a center-filled gum, a confectionary, including mints, lozenges.

Aspects of the invention relate to methods for delivery of particles to bone. In some embodiments the method comprises administering to a subject a calcium particle linked to both a modifying ligand and a hydroxyapatite binding agent, to deliver the calcium particle to the bone. In some embodiments the modifying ligand comprises a stealth ligand such as poly(ethylene glycol), hyaluronic acid, dextran, chitosin, or poly(ethylene oxide). In certain embodiments the particle further comprises an agent, factor or drug. In some embodiments the agent, factor or drug is incorporated within the particle through diffusion into pores within the particle.

In some embodiments the agent comprises an agent to promote bone growth. In certain embodiments the agent comprises a growth factor or a cytokine such as leptin, sortilin, transglutaminase, prostaglandin E, 1,25-dihydroxyvitamin D3, ascorbic acid, β-glycerol phosphate, TAK-778, statins, interleukins such as IL-3 and IL-6, growth hormone, steel factor (SF), activin A (ACT), retinoic acid (RA), epidermal growth factor (EGF), bone morphogenctic proteins (BMP), platelet derived growth factor (PDGF), hepatocyte growth factor, insulin-like growth factors (IGF) I and II, hematopoietic growth factors, peptide growth factors, erythropoietin, interleukins, tumor necrosis factors, interferons, colony stimulating factors, heparin binding growth factor (HBGF), alpha or beta transforming growth factor (α or β-TGF) such as TGF-β1, fibroblast growth factors, vascular endothelium growth factor (VEGF), nerve growth factor (NGF) and muscle morphogenic factor (MMP).

In some embodiments the particle comprises a factor that promotes the production or assembly of collagen such as pro-collagen or ascorbic acid. In certain embodiments the particle comprises a resorption factor for promoting particle resorption into bone such as receptor activator of NFκB ligand (Rank-L), a cortitosteroid such as dexamethasone, a parathyroid hormone, macrophage colony stimulating factor (M-CSF), or transforming growth factor-β1 (TGF-β1). In some embodiments the particle comprises an agent that chelates minerals from blood such as an EDTA-based agent, poly(bisphosphonate), poly(phosphate), biological or non biological entities that nucleate calcium and/or phosphate, aspartic acid, osteopontin, or bone sialoprotein.

In some embodiments the particle further comprises a linker attaching the modifying ligand to the particle. In certain embodiments the linker comprises poly(ethylene glycol), hyaluronic acid, dextran, chitosan, or poly(ethylene oxide).

In some embodiments the method of administering the particle to a subject comprises oral, intravenous injection, subcutaneous injection, parenteral, rectal, sublingual, transdermal, nasal, inhalation, and ocular administration. In some embodiments the method of administering the particle to a subject comprises injection of the particle into bone. In certain embodiments the particle is incorporated into a delivery vehicle to control the release of the particle into the bloodstream and/or bone. In some embodiments the delivery vehicle comprises a degradable polymer such as poly hydroxy acids such as poly(lactide-co-glycolide) (PLGA), dextran, hyaluronic acid, poly(citrate), poly(glycerol sebacate), chitosan, elastin, or poly(carbonate).

In some embodiments the particle comprises a drug for treatment of osteoporosis such as bisphosphonate based drugs, estrogen receptor modulators, or hormone based therapies. In certain embodiments the particle comprises a drug that inhibits osteoclast resorption such as raloxifene, AAR494, or E-64. In some embodiments the method further comprises a method for treating osteopenia. In some embodiments the method further comprises a method for treating bone cancer. In some embodiments the particle comprises a bone targeting factor such as a granulocyte colony-stimulating factor, or a bone marrow specific membrane surface receptor. In some embodiments the bone targeting factor is a factor such as pentosidine that targets osteoporotic bone. In some embodiments the method further comprises a method for targeting the bone marrow space.

Aspects of the invention relate to treating osteoporosis through delivery of calcium particles to bone. In some embodiments of the invention the method for treating osteoporosis comprises administering to a subject having or at risk of having osteoporosis an effective amount of a calcium particle linked to both a modifying ligand and a hydroxyapatite binding agent to treat osteoporosis in the subject. In some embodiments the modifying ligand comprises a stealth ligand such as poly(ethylene glycol), hyaluronic acid, dextran, chitosin, or poly(ethylene oxide).

In certain embodiments the particle further comprises an agent, factor or drug. In some embodiments the agent, factor or drug is incorporated within the particle through diffusion into pores within the particle.

In some embodiments the agent comprises an agent to promote bone growth. In certain embodiments the agent comprises a growth factor or a cytokine such as leptin, sortilin, transglutaminase, prostaglandin E, 1,25-dihydroxyvitamin D3, ascorbic acid, β-glycerol phosphate, TAK-778, statins, interleumins such as IL-3 and IL-6, growth hormone, steel factor (SF), activin A (ACT), retinoic acid (RA), epidermal growth factor (EGF), bone morphogenetic proteins (BMP), platelet derived growth factor (PDGF), hepatocyte growth factor, insulin-like growth factors (IGF) I and II, hematopoietic growth factors, peptide growth factors, erythropoietin, interleukins, tumor necrosis factors, interferons, colony stimulating factors, heparin binding growth factor (HBGF), alpha or beta transforming growth factor (α or β-TGF) such as TOF-β1, fibroblast growth factors, vascular endothelium growth factor (VEGF), nerve growth factor (NGF) and muscle morphogenic factor (MMP).

In some embodiments the particle comprises a factor that promotes the production or assembly of collagen such as pro-collagen or ascorbic acid. In certain embodiments the particle comprises a resorption factor for promoting particle resorption into bone such as receptor activator of NFκB ligand (Rank-L), a cortitosteroid such as dexamethasone, a parathyroid hormone, macrophage colony stimulating factor (M-CSF), or transforming growth factor-β1 (TGF-β1). In some embodiments the particle comprises an agent that chelates minerals from blood such as an EDTA-based agent, poly(bisphosphonate), poly(phosphate), biological or non biological entities that nucleate calcium and/or phosphate, aspartic acid, osteopontin, or bone sialoprotein.

In some embodiments the particle further comprises a linker attaching the modifying ligand to the particle. In certain embodiments the linker comprises poly(ethylene glycol), hyaluronic acid, dextran, chitosan, or poly(ethylene oxide).

In some embodiments the method of administering the particle to a subject comprises oral, intravenous injection, subcutaneous injection, parenteral, rectal, sublingual, transdermal, nasal, inhalation, and ocular administration. In some embodiments the method of administering the particle to a subject comprises injection of the particle into bone. In certain embodiments the particle is incorporated into a delivery vehicle to control the release of the particle into the bloodstream and/or bone. In some embodiments the delivery vehicle comprises a degradable polymer such as poly hydroxy acids such as poly(lactide-co-glycolide) (PLGA), dextran, hyaluronic acid, poly(citrate), poly(glycerol sebacate), chitosan, elastin, or poly(carbonate).

In some embodiments the particle comprises a drug for treatment of osteoporosis such as bisphosphonate based drugs, estrogen receptor modulators, or hormone based therapies. In certain embodiments the particle comprises a drug that inhibits osteoclast resorption such as raloxifene, AAR494, or E-64. In some embodiments the method further comprises a method for treating osteopenia. In some embodiments the method further comprises a method for treating bone cancer. In some embodiments the particle comprises a bone targeting factor such as a granulocyte colony-stimulating factor, or a bone marrow specific membrane surface receptor. In some embodiments the bone targeting factor is a factor such as pentosidine that targets osteoporotic bone.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 demonstrates layer-by-layer (LBL) formulation of calcium carbonate (CaCO₃)-alginate-poly-L-lysine(PLL) particles on a glass surface.

FIG. 2 demonstrates layer-by-layer (LBL) formulation of CaCO₃-alginate-PLL-alginate particles on an alginate coated glass surface.

FIG. 3 demonstrates layer-by-layer (LBL) formulation of alginate and PLL on an alginate-coated glass surface.

FIG. 4 demonstrates the separation of the unbound PLL-FITC from the solution containing CaCO₃-alginate-PLL particles.

FIG. 5 demonstrates the binding of CaCO₃-carboxymethyl cellulose (CMC)-PLL and CaCO₃-poly polystyrene sulfonic acid (PSS)-PLL particles to a glass surface coated with 0.01% of Poly-L-Lysine-FITC.

FIG. 6 is a schematic demonstrating the layer-by-layer deposition of alternating charges of coating to a biological substrate (A), which upon hydrolysis will displace the layers from the surface.

FIG. 7 depicts another example of the layer-by-layer approach.

FIG. 8 depicts another example of the layer-by-layer approach whereby layers are built up on a substrate prior to application to the biological surface.

FIG. 9 depicts the formation of protein-filled capsules.

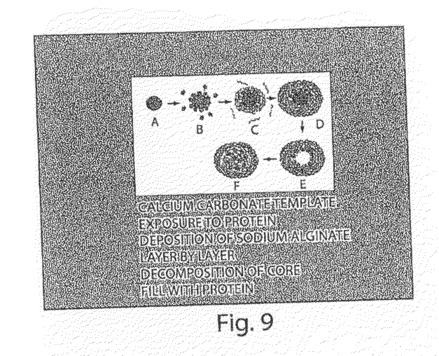

FIG. 10 is a schematic depicting certain embodiments of the invention.

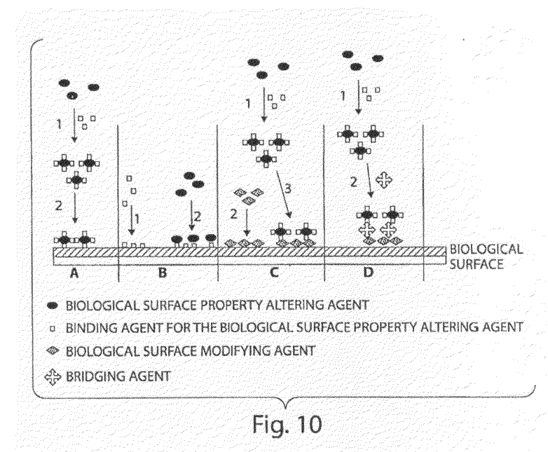

FIG. 11 is a graph demonstrating aggregation of CaCO₃ particles in a solution containing bisphosphonate-poly(D,L-lactide-co-glycolide)-co-poly(ethylene glycol) (PLGA-PEG) nanoparticles.

FIG. 12 depicts a kit with particles according to an embodiment of the invention (10=kit, 12=container of particles, 14=container of additional components, 20=instructions).

FIG. 13 is a diagram of several commercially available forms of bisphosphonates.

### DETAILED DESCRIPTION

The invention relates to methods and compositions for delivering particles to structures such as tooth or bone. The particles can be used to modify the surface of the structure and/or to deliver active agents to the structure. For instance, the particle based technologies may be used for the purpose of applying an agent to a tooth or in proximity of the tooth. The agent can be used, for instance for the purpose of tooth whitening or other coloring, masking stains on teeth, preventing stains or bacterial adhesion to the teeth, treating tooth or gum disorders such as carries and halitosis, remineralization of enamel, reducing plaque formation, and bacteria reduction in the oral cavity. Methods for creating and/or modifying nanoparticles and microparticles for such applications are described herein. The particles, which may be delivered to the tooth surface or within the tooth itself, may have a natural bright white appearance, for instance, when it is desirable to whiten teeth. The particles may be designed to briefly coat the tooth surface for rapid-acting, short lasting activity, to bond to the tooth surface for various desirable durations, or to be entrapped within the pores of the tooth for longer term “deep” whitening or other therapies. Covalent bonds can be formed with the proteins or other molecules or components such as bacteria present on the surface of the teeth in order to facilitate a temporary bond with the tooth surface. Alternatively through applying a strongly adherent coating to the tooth surface, (e.g. based on acrylate chemistry), the duration of effect may be increased through use of covalent bonds to the applied coating. To obtain shorter life times, non-covalent ionic bonds may be used.

The methods and compositions provided herein also include particle based technologies for delivering calcium and/or other agents to bone or bone marrow with applications for treatment of bone disorders. The particles can be designed to target to the bone in order to deliver an active agent, such as calcium. The design of the particle will effect the amount of particles which will be delivered to the bone and maintained at the bone surface to deliver the calcium. Optimal designs are discussed below. The local release of calcium and optionally other agents at the bone surface provides a useful advantage for the treatment of bone disorders.

The compositions useful according to the methods of the invention are composed at a minimum of a particle. The particles may be modified to adhere (physically or chemically) to a biological surface i.e. tooth or bone or to bacteria that coat the surface. Alternatively the particles may inherently have this property and may not require further manipulation. When the particles are modified, the modification may be, for instance, in the form of layer-by-layer deposition of polymeric materials on the surfaces or by chemical conjugation of molecules that induce adhesion of the particles to the surfaces. Such molecules are referred to as binding agents because they have the ability to bind to the biological surface or a coating on the biological surface. The binding agent may be linked directly or indirectly to the particle through a linker. Additionally, the compositions may include active agents such as biological agents, fillers, preservatives etc.

As used herein the term “particle” includes nanoparticles as well as microparticles. Nanoparticles are defined as particles of less than 1.0 μm in diameter. A preparation of nanoparticles includes particles having an average particle size of less than 1.0 μm in diameter. Microparticles are particles of greater than 1.0 μm in diameter but less than 1 mm. A preparation of microparticles includes particles having an average particle size of greater than 1.0 μm in diameter. The microparticles may therefore have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. A composition of particles may have heterogeneous size distributions ranging from 10 nm to mm sizes. In some embodiments the diameter of the altering agent is about 5 nm to about 500 nm. In other embodiments, the diameter is about 100 nm to about 200 nm. In other embodiment, the diameter of the altering agent is about 10 nm to about 100 nm.

The particles may be composed of a variety of materials including ceramic, metallic, natural polymer materials (including lipids, sugars, chitosan, hyaluronic acid etc), synthetic polymer materials (including poly-lactide-coglycolide, poly-glycerol sebacate, etc), and non-polymer materials, or combinations thereof.

The particles may be composed in whole or in part of polymers or non-polymer materials. Non-polymer materials, for example, may be employed in the preparation of the particles. Exemplary materials include alumina, calcium carbonate, calcium sulfate, calcium phosphosilicate, sodium phosphate, calcium aluminate, calcium phosphate, hydroxyapatite, tricalcium phosphate, dicalcium phosphate, tricalcium phosphate, tetracalcium phosphate, amorphous calcium phosphate, octacalcium phosphate, and silicates. In certain embodiments the particles may comprise a calcium salt such as calcium carbonate, a zirconium salt such as zirconium dioxide, a zinc salt such as zinc oxide, a magnesium salt such as magnesium silicate, a silicon salt such as silicon dioxide or a titanium salt such as titanium oxide or titanium dioxide.

A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621; 4,638,045 to Kohn; see also Langer, Ace. Chem. Res. 33:94, 2000; Langer, J. Control Release 62:7, 1999; and Uhrich et al., Chem. Rev. 99:3181, 1999; all of which are incorporated herein by reference).

Polymers include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride and polystyrene.

Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as algninate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In some embodiments the polymers are polyesters, polyanhydrides, polystyrenes, polylactic acid, polyglycolic acid, and copolymers of lactic and glycoloic acid and blends thereof.

PVP is a non-ionogenic, hydrophilic polymer having a mean molecular weight ranging from approximately 10,000 to 700,000 and the chemical formula (C₆H₉NO)[n]. PVP is also known as poly[1-(2-oxo-1-pyrrolidinyl)ethylene], Povidone™, Polyvidone™, RP 143™, Kollidon™, Peregal ST™, Periston™, Plasdone™, Plasmosan™, Protagent™, Subtosan™, and Vinisil™. PVP is non-toxic, highly hygroscopic and readily dissolves in water or organic solvents.

Polyethylene glycol (PEG), also known as poly(oxyethylene) glycol, is a condensation polymer of ethylene oxide and water having the general chemical formula HO(CH₂CH₂O)[n]H.

Polyvinyl alcohol (PVA) is a polymer prepared from polyvinyl acetates by replacement of the acetate groups with hydroxyl groups and has the formula (CH₂CHOH)[n]. Most polyvinyl alcohols are soluble in water.

PEG, PVA and PVP are commercially available from chemical suppliers such as the Sigma Chemical Company (St. Louis, Mo.).

In certain embodiments the particles may comprise poly(lactic-co-glycolic acid) (PLGA).

In some embodiments, the particles comprise calcium particles. As used herein calcium particles refer to any particles that contain calcium. For example in certain embodiments the particle may have at least 0.1%, at least 1%, 5%, at least 10%, at least 20%, at least 30%, at least 40%, at least 50%, at least 60%, at least 70%, at least 80%, or at least 90% calcium (% wt). In some embodiments a calcium particle may comprise a calcium salt, polymer, ceramic, biopolymer, or composite, and may be in the form of a nanoparticle, microparticle, aggregate, assembly, or micelle. In some embodiments calcium particles comprise calcium salts such as calcium carbonate, calcium phosphate, calcium sulphate, calcium citrate, calcium oxalate, calcium malate, calcium gluconate, calcium silicate, or calcium stearate.

In certain embodiments calcium particles comprise calcium carbonate. Calcium carbonate is a safe, non-toxic and inexpensive material that is easy to colorize by adding pigments, if desired, and can be functionalizable by formulating with other components as described herein. It also has the beneficial property of remineralizing teeth. Calcium carbonate is currently used industrially as paper fillers (to improve texture, appearance and ink absorption), coatings (for example, weather resistance and anti-corrosion), as a component of plastics (to increase mechanical properties), in agriculture (as a fertilizer, feed stock and soil pH adjuster), and in the health care field as an abrasive in tooth pastes and in calcium supplements. Calcium carbonate is available in several morphologies, such as scalenohedral calcite precipitated calcium carbonate (PCC), spherical calcite PCC, prismatic calcite PCC, clustered acicular aragonite PCC, rhombohedral calcite PCC and discrete acicular aragonite PCC, by way of non-limiting examples. Each of these forms of calcium carbonate has applicability in the teachings herein, selection thereof dependent on desirable altering of color, texture, mouth feel, and other properties of a biological surface as described herein. In some embodiments, a calcium carbonate nanoparticle is used for tooth whitening, since it can provide the desired degree of whiteness, texture and shine.

In some embodiments, the particles may consist of both organic and inorganic components. In certain embodiments, the particles may comprise a hydrogel containing calcium. In some embodiments, the hydrogel containing calcium is calcium crosslinked alginate.

In some embodiments, the particles may consist of inorganic shells that may be doped with drugs or growth factors. Through use of sacrificial templates such as calcium carbonate (CaCO(₃)) microparticles coupled with a two-step deposition of polyelectrolyte coatings by surface controlled precipitation (SCP) followed by the layer-by-layer (LbL) adsorption technique, capsule shells can be obtained. When sodium alginate is used for inner shell assembly, template decomposition with an acid results in simultaneous formation of microgel-like structures due to calcium ion-induced gelation. An extraction of the calcium after further LbL treatment results in microcapsules filled with the biopolymer. The density of these particles can be manipulated through filling the core with different agents.

Coating the calcium carbonate particles with a thin layer may help facilitate functionalization of the particles. Such layers may include but are not limited to alginate which can easily be precipitated onto the surface.

The particles may be formulated in a solution or suspension. As such, the majority of the particles may not be in physical contact with many other particles, for instance, more than two particles. Preferably, the majority of particles is not in contact with more than one other particle. And even more preferably, the majority of particles is not in contact with any other particle. As used herein, a majority of particles means greater than 50%, including at least 55%, at least 60%, at least 65%, at least 70%, at least 75%, at least 80%, at least 85%, at least 90%, or at least 95% of particles. The particles in solution, as used herein, therefore are not compacted and do not form a matrix.

In some embodiments, the particles are porous. A porous particle can be a particle having one or more channels that extend from its outer surface into the core of the microchannel. In some embodiments, the channel may extend through the particle such that its ends are both located at the surface of the particle. These channels are typically formed during synthesis of the particle by inclusion followed by removal of a channel forming reagent in the particle.

The size of the pores may depend upon the size of the particle. In certain embodiments, the pores have a diameter of less than 15 microns, less than 10 microns, less than 7.5 microns, less than 5 microns, less than 2.5 microns, less than 1 micron, less than 0.5 microns, or less than 0.1 microns. The degree of porosity in porous particles may range from greater than 0 to less than 100% of the particle volume. The degree of porosity may be less than 1%, less than 5%, less than 10%, less than 15%, less than 20%, less than 25%, less than 30%, less than 35%, less than 40%, less than 45%, or less than 50%. The degree of porosity can be determined in a number of ways. For example, the degree of porosity can be determined based on the synthesis protocol of the carriers (e.g., based on the volume of the aqueous solution or other channel-forming reagent) or by microscopic inspection of the carriers post-synthesis.

The plurality of particles may be homogeneous for one or more parameters or characteristics. A plurality that is homogeneous for a given parameter, in some instances, means that particles within the plurality deviate from each other no more than about +/−10%, preferably no more than about +/−5%, and most preferably no more than about +/−1% of a given quantitative measure of the parameter. As an example, the particles may be homogeneously porous. This means that the degree of porosity within the particles of the plurality differs by not more than +/−10% of the average porosity. In other instances, a plurality that is homogeneous means that all the particles in the plurality were treated or processed in the same manner, including for example exposure to the same agent regardless of whether every particle ultimately has all the same properties. In still other embodiments, a plurality that is homogeneous means that at least 80%, preferably at least 90%, and more preferably at least 95% of particles are identical for a given parameter.

The plurality of particles may be heterogeneous for one or more parameters or characteristics. A plurality that is heterogeneous for a given parameter, in some instances, means that particles within the plurality deviate from the average by more than about +/−10%, including more than about +/−20%. Heterogeneous particles may differ with respect to a number of parameters including their size or diameter, their shape, their composition, their surface charge, their degradation profile, whether and what type of agent is comprised by the particle, the location of such agent (e.g., on the surface or internally), the number of agents comprised by the particle, etc. The invention contemplates separate synthesis of various types of particles which are then combined in any one of a number of pre-determined ratios prior to contact with the sample.

As an example, in one embodiment, the particles may be homogeneous with respect to shape (e.g., at least 95% are spherical in shape) but may be heterogeneous with respect to size, degradation profile and/or agent comprised therein. In some embodiments the particles form an aggregate. The aggregate of particles may be a homogenous or heterogeneous plurality of particles as described above. In some embodiments, the diameter of the aggregate of particles may be less than about 500 nm. In other embodiments, the diameter of the aggregate of particles may be about 1 μm. In other embodiments, the diameter of the aggregate of particles may be about 500 nm to about 10 μm. In other embodiments, the diameter of the aggregate of particles may be about 10 μm to about 100 μm; in still other embodiments, the diameter of the aggregate of particles mat be about 100 μm to about 500 μm.

Particle size, shape and release kinetics can also be controlled by adjusting the particle formation conditions. For example, particle formation conditions can be optimized to produce smaller or larger particles, or the overall incubation time or incubation temperature can be increased, resulting in particles which have prolonged release kinetics.

The particles may also be coated with one or more stabilizing substances, which may be particularly useful for long term depoting with parenteral administration or for oral delivery by allowing passage of the particles through the stomach or gut without dissolution. For example, particles intended for oral delivery may be stabilized with a coating of a substance such as mucin, a secretion containing mucopolysaccharides produced by the goblet cells of the intestine, the submaxillary glands, and other mucous glandular cells.

Additionally, the particles can be non-covalently or covalently coated with compounds such as fatty acids or lipids. The coating may be applied to the particles by immersion in the solubilized coating substance, spraying the particles with the substance or other methods well known to those skilled in the art. The particles may be incorporated, for instance, into liposomes, virosomes, cationic lipids or other lipid based structures. The term “cationic lipid” refers to lipids which carry a net positive charge at physiological pH. Such lipids include, but are not limited to, DODAC, DOTMA, DDAB, DOTAP, DC-Chol and DMRIE. Additionally, a number of commercial preparations of cationic lipids are available. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA). A variety of methods are available for preparing liposomes e.g., U.S. Pat. Nos. 4,186,183, 4,217,344, 4,235,871, 4,261,975, 4,485,054, 4,501,728, 4,774,085, 4,837,028, 4,946,787; and PCT Publication No. WO 91/17424.

The particles may also be composed in whole or in part of GRAS components. i.e., ingredients are those that are Generally Regarded As Safe (GRAS) by the US FDA. GRAS components useful as particle material include non-degradeable food based particles such as cellulose. Other suitable GRAS or FDA approved materials that may be useful in application to the teeth or bones include anticaries drugs, fluoride, sodium fluoride, sodium monofluorophosphate, stannos fluoride, hydrogen peroxide, carbamide peroxide (i.e., urea peroxide), antibacterial agents, plaque removing agents, stain removers, anticalculus agents, abrasives, baking soda, perearbonates, perborates of alkali and alkaline earth metals, or similar type substances, or combinations thereof. The FDA maintains several non all-inclusive lists of GRAS substances. These lists are maintained in 21 CFR Part 182, 21 CFR Part 184, and 21 CFR Part 186, each of which is incorporated by reference.

The particles useful according to the invention may including a binding agent. As used herein the term binding agent refers to any agent that promotes or facilitates binding of a particle to a desired organ, tissue or cell of the body. In certain embodiments a binding agent may target a particle to a biological surface such as a tooth or a bone. The binding agent may bind directly to the biological surface or materials coating the biological surface. For instance, the biological surface may include a natural coating such as bacteria on the teeth or the biological surface may first be modified by applying a coating to the surface such that a binding agent will bind to the biological surface. In general binding agents useful according to the invention will be those that target or bind to a surface of a tooth, a surface of the gum or surrounding tissue in the oral cavity, a component inside of a tooth, the surface of a bone, the bone marrow space, or domains of trabecular bone where growth and remodeling happens with regularity.

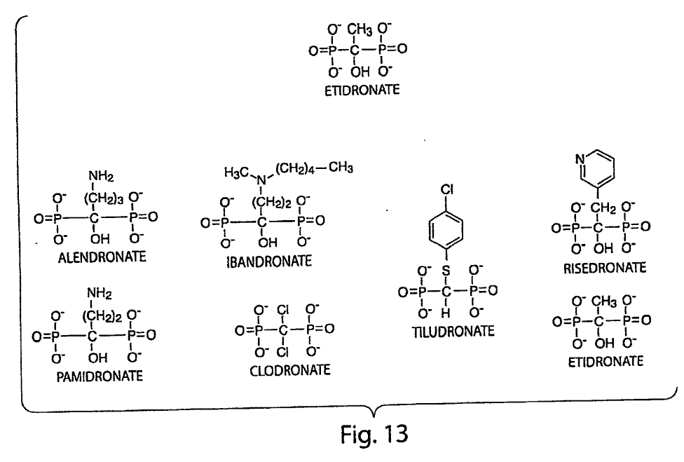

Hydroxyapatite (a form of calcium phosphate) is a major component of both teeth and bones. Thus in certain embodiments of the invention, the binding agent may be a hydroxyapatite binding agent. As used herein hydroxyapatite binding agent refers to any agent that is capable of binding to or otherwise exhibiting a chemical affinity for hydroxyapatite. The term binding refers to adhering either biologically or chemically to a biological surface. In some embodiments the hydroxyapatite binding agent has an affinity for hydroxyapatite with a Kd of less than 10⁻⁶ M. In some embodiments, the hydroxyapatite binding agent has an affinity for hydroxyapatite with a Kd of less than 10⁻⁸ M. In some embodiments a hydroxyapatite binding agent is capable of binding to any surface or composition that contains hydroxyapatite such as hydroxyapatite within a tooth or within a bone. In certain embodiments the hydroxyapatite binding agent is a bisphosphonate. Bisphosphonates all contain two phosphonic acid groups on the same carbon. One of the most common examples is the sodium salt of etidronate. Structures of etidronate and other commercially available bisphosphonates are shown in FIG. 13. Bisphosphonates are characterized by two carbon-phosphorous bonds, the carbon atom replacing the oxygen in the P—O—P (phosphorous-oxygen-phosphorous) bond of pyrophosphate and the P—C—P bond conferring resistance to chemical and enzymatic hydrolysis. Different substitutions on the carbon atom have created several different bisphosphonates, each with its own pharmacological properties. Etidronate, contains a hydroxyl and methyl group substitution on the carbon atom, resulting in a bisphosphonate with a half-life in bone of greater than 90 days. Other more potent bisphosphonates have subsequently been developed, such as alendronate, which has an alkyl amine and hydroxyl group substitution on the carbon atom. The side chains of some analogs of bisphosphonates may be modified to create bisphosphonates with altered properties which may be useful according to the invention. In some embodiments the bisphosphonate may be Alendronte (Fosamax), Risedronate (Actonel), or Ibandronate sodium (Boniva).

The strong affinity of bisphosphonates, and other hydroxyapatite-targeting moieties, for teeth and bone allows the compositions of the invention to be effective targeting delivery systems for biologically active agents.

Hydroxyapatite binding agents may also include tetracycline, calcein, polyaspartic acid, polyglutamic acid, or aminophosphosugars as well as anti-hydroxyapatite antibodies and natural or synthetic binding peptides. In some embodiments the hydroxyapatite binding agent may comprise an agent found in saliva.

In some embodiments a hydroxyapatite binding agent may comprise a cationic polymer. In certain embodiments the cationic polymer may be PLL or PEI. In some embodiments the cationic polymer may be selected from cationic GRAS components such as those listed by the FDA, maintained in 21 CFR Part 182, 21 CFR Part 184, and 21 CFR Part 186, each of which is incorporated by reference.

In certain embodiments a binding agent that targets a particle to a tooth may bind to saliva on a tooth, and could comprise any agent that binds to saliva. In other embodiments a binding agent that targets a particle to a tooth may bind to bacteria on the surface of the tooth and could comprise any agent that is capable of binding to bacteria found on the surface of a tooth. A particle may have specificity to bind to a particular biological surface, such as to teeth and not mucosa, or to mucosa and not teeth, and thus would be targeted to that surface from the means of delivery, for example, a mouthwash.

In certain embodiments, a binding agent may be an ionic agent. Selections of ionic agents include anionic and cationic agents, either of which may be polymeric or nonpolymeric, or oligomeric. In one embodiment, the anionic agent is a nonpolymeric anionic agent, such as a fatty acid, for example, stearic acid. In another embodiment, the nonpolymeric anionic agent is bisphosphonate. In another embodiment, the anionic agent is a polymeric anionic agent such as a poly(carboxylic acid), poly(phosphonic acid) or polyphosphate. In another embodiment, the poly(carboxylic acid) is polyacrylic acid or alginic acid. In another embodiment, polymeric anionic agent is a polyphosphonated or polymer or polysulfate, such as polyphosphonated polyethylene or carrageenan. In another embodiment the polymeric anionic agent is a polysulfonic acid such as polystyrene sulfonic acid. In another embodiment, the anionic polymer is DNA, peptides, heparin and sugars/glycosaminoglycans (GAGs). In another embodiment, the anionic agent is an anionic dendrimer such as PAMAM dendrimer, a polypropyleneimine (DAB-AM) dendrimer or a phosphorus-based dendrimer. In another embodiment, the anionic agent is an anionic oligomer such as an oligonucleotide or dextran sulfate. In another embodiment the anionic oligomer is a water-soluble or water-dispersible polymer comprising one or more monomers selected from an unsaturated carboxylic acid and a derivative thereof, or a water-soluble or water-dispersible polymer comprising an unsaturated sulfonic acid or a derivative thereof.

The binding agent alternatively may be an acrylate, such as cyanoacrylate. A composition comprising a particle and an acrylate binding agent provides a means for adherence of the particle to the biological surface, directly or in embodiments where a linker is also employed. In another embodiment the binding agent is selected from dental resins, such as glass ionomer, zinc phosphate cement, or Bis-GMA.

The binding agent may also be cationic. Examples of cationic binding agents include polymeric and nonpolymeric cationic agents. In one embodiment, the binding agent is chitosan or a polyamine. The composition comprising the binding agent and the particle may bind directly to the biological surface or indirectly via a bridging agent or biological surface modifying agent.

In certain embodiment the binding agent may bind specifically to osteoporotic bone. One example of a binding agent for osteoporotic bone is pentosidine. Other binding agents include zanthan gum, pectin, pullulan, guar gum, agar, polyvinyl pyrollidone (PVP), carrageenan, starch, flour, mussel adhesive protein, an oxidation product of 3,4dihydroxyphenylalanine, a cellulose, a silicone resin, such as an organosiloxane resin, a chelating agent such as a pyrophosphate, triphosphate, polyphosphate, polyphosphonate, dialkali metal pyrophosphate salt, tetraalkali polyphosphate salt, EDTA-related polymer, a polyol, glycerin, propylene glycol, polypropylene glycol, polyethylene glycol, sorbitol, xylitol, copolymer, such as acrylic acid cross-linked with polyallyl sucrose, an organic polymer acid colloid, a polyuronic acid, a carboxypolymethylene compound, a polyester resin containing three carboxyl groups, a partially hydrolyzed polyacrylate, polymethacrylates, polyoxyethylenes, polypropylene copolymers, and a polysaccharide such as cellulose, ethylcellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, starch, or flour. In another embodiment, the binding agent is one of known binding pair such as avidin and biotin.

Molecules, distinct from the macromolecules of which the particles are composed, may be attached to the outer surface of the particles by methods known to those skilled in the art to “coat” or “decorate” the particles. The molecules may be attached directly or indirectly to the outer surface of the particle for instance through the use of a linker (discussed below). These molecules are attached for purposes such as to facilitate binding, enhance receptor mediation, and provide escape from endocytosis or destruction. For example, biomolecules such as phospholipids may be attached to the surface of the particle to prevent endocytosis by endosomes; receptors, antibodies or hormones may be attached to the surface to promote or facilitate binding of the particle to the desired organ, tissue or cells of the body; and polysaccharides, such as glucans, or other polymers, such as polyvinyl pyrrolidone and PEG, may be attached to the outer surface of the particle to enhance or to avoid uptake by macrophages.

A linker is a molecule that is capable of being attached to the particles directly or indirectly. As used herein, the term “linked” refers to any combination of two or more component parts that are linked together, directly or indirectly, via any physicochemical interaction. In one embodiment the linkage is a combination of two or more component parts that are linked together, directly or indirectly, via covalent bonding. A particle and binding agent are “linked directly” if they are covalently or non-covalently bound to one another with no intervening structures. The particle and binding agent are said to be “linked indirectly” if they are connected to one another via a linker.

Another molecule can also be attached to the linker. Linkers useful according to the invention include but are not limited to amine, methylamine, carboxylic acid, maleimide-succinimide, and maleimide-hydrazine linkers. Linkers may include functional groups such as a hydroxyl group, a primary or secondary amino group, a phosphate group or substituted derivatives thereof or a carboxylic acid group. Polar lipids such as acyl carnitine, acylated carnitine, sphingosine, ceramide, phosphatidyl choline, phosphatidyl glycerol, phosphatidyl ethanolamine, phosphatidyl inositol, phosphatidyl serine, cardiolipin and phosphatidic acid may also function as linkers. The polar lipid molecules may optionally be covalently linked to an organic spacer molecule which may or may not have functional groups. Other linkers include heterobifunctional cross-linkers. For instance, succinimidyl-4-(N-maleimidomethyl)cyclohexane-1-(carboxy-6-aminocaproate)-, also known as LC-SMCC, is a heterobifunctional cross-linker that reacts with sulfhydryl group and amine-groups. Non-limiting examples of linkers include the polymeric anionic, cationic and nonionic agents described above.

The linker may be attached to a binding agent, for instance, for the purpose of targeting the particle to a particular tissue or cell. In some embodiments the binding agent is a targeting agent such as a glycoprotein, an antibody, or a binding protein.

The linker connecting the particle and binding agent may contain an enzyme susceptible site. An “enzyme susceptible site” as used herein refers to a portion of a molecule that is recognized and cleaved by an enzyme. In some embodiments, the enzyme susceptible site is recognized and cleaved by an enzyme naturally present in the target cell.

In some embodiments the binding agent or the linker is conjugated to a functional group of the particle. Alternatively, the particle or the linker is conjugated to a functional group of the binding agent.

In certain embodiments a property of the particle may be modified to produce a composition with another property. Properties of the particle that may be modified include but are not limited to surface charge, relative hydrophobicity/hydropholicity, and surface texture. In some embodiments a particle may be modified through attaching biological (antibodies, peptides, nucleotides) or synthetic (small molecules, aptamers) molecules. Similar techniques can also be used to control the timing or location of activity. Particles may also be combined with drugs (including antimicrobials, anti-viral agents, anti-neoplastic agents, anesthetics) or factors to promote a desired therapeutic outcome (including reduction in bacteria, treatment of fungal infections, breath freshening, calcium binding to prevent calcification, reduction in sensitivity). Functionalities rendered to the particle surface (including amine, hydroxyl, carboxyl, or fluoride groups) can improve attachment to the biological surface. In some embodiments particles may be combined with silica and/or sodium hexametaphosphate for increased whitening effects. Antimicrobial effects may also be achieved through use of “self sterilizing materials”. These materials include quaternary ammonium compounds. Poly(4-vinylpyridine) (PVP) can be used as a carrying polymer with N-alkylation, where the resultant poly(4-vinyl-N-alkylpyridinium bromide) has antimicrobial effects. Covalent immobilization of alkylated polyethylenimine and other moieties also may be used for this purpose.

In certain embodiments, binding between a particle and a biological surface may involve an avidin biotin complex. One member of the avidin biotin complex may be associated with the particle, and the other with the biological surface, either directly or indirectly.

In some embodiments, to improve adhesion to the biological surface, materials typically used for surgical glues, such as cyanoacrylate, may be applied to the surface or to the particle. These materials may be naturally white, contain a coloring agent, contain colored particles, or be combinations thereof. These materials would typically be useful for increasing the length of temporary tooth whitening. In some embodiments, materials such as sodium polyacrylate, including low or high molecular weight forms, may be used to enhance bonding of a particle to the tooth surface. In some embodiments, materials that enhance binding may be used on the biological surface to improve adhesion of particles. In other embodiments, materials that enhance binding may be incorporated with the particles into a resin before being applied to the biological surface.

The bonding duration will be controlled by the properties of the bonding. Covalent bonds can be formed with the surfaces of the proteins on the teeth in order to facilitate durations comparable to the duration that proteins typically remain on the tooth surface. Alternatively through applying a strongly adherent coating to the tooth surface, (e.g. based on acrylate chemistry), the duration of effect may be increased through use of covalent bonds to the applied coating. To obtain shorter life times, non-covalent ionic bonds may be used.

This particle based technology may also be useful to combine with degradable materials for biomedical applications. For example, particles may be incorporated into tissue engineering scaffolds or applied to their surfaces for release of drugs and/or for improved mechanical properties.

In certain embodiments the particles may comprise one or more agents. The agents may be located (e.g., incorporated) within the particle (e.g., within pores or channels of the particle) and/or on the external surface of the particle. In some instances, the particles are pre-loaded with the one or more agents. The agents include but are not limited to biological agents including drugs such as growth factors, anti-microbial agents, inhibitors of bone resorption, flavoring agents, bleaching agents, sweetening agents, antioxidants, saliva stimulating agents, breath freshening agents, antiplaque agents, anti-inflammatory agents, H2 antagonists, desensitizing agents, nutrient, biomolecules, pacifying agents, whitening (coloring agents), additional pigments, fluoride ion sources, abrasive agents, analgesics, surfactants, humectants, tartar control agents, detergents, thickeners, or preservatives.

Biological agents include diagnostic, cosmetic, and therapeutic agents, such as releasable drugs. Thus, any biological agent can be incorporated within the particles, which can locally or systemically deliver or maintain the incorporated agents following administration or application to a subject. Any biocompatible or pharmacologically acceptable material can be incorporated into the particles or trapped in the pores of the particles using technology known to those skilled in the art. Biological agents include but are not limited to synthetic inorganic and organic compounds, proteins and peptides, polysaccharides and other sugars, lipids, and DNA and RNA nucleic acid sequences having therapeutic, prophylactic, cosmetic or diagnostic activities. Nucleic acid sequences include genes, plasmids, vectors, antisense molecules that bind to complementary DNA to inhibit transcription, siRNA, shRNA, and ribozymes.

In certain instances, the biological agent is an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics that are effective against a single organism or disease and not against other types of bacteria, are referred to as limited spectrum antibiotics. Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Inhibitors of bone resorption include but are not limited to proton pump inhibitors, bisphosphonates, e.g. clodronic acid, etidronic acid, pamidronic acid, aledronic acid, ibandronic acid, zoledronic acid, risedronic acid or tiludronic acid, and salts and hydrates thereof, steroid hormones e.g. estrogen, a partial estrogen agonist or estrogen-gestagen combination, a SERM (Selective Estrogen Receptor Modulator) e.g. raloxifene, lasofoxifene, TSE-424, FC1271, Tibolone (LIVLAL®), tamoxifene, droloxifene, toremifene, idoxifene, or levormeloxifene), calcium, a calcitonin-like substance or derivative thereof, e.g. salmon, eel or human calcitonin, vitamin D or an analog thereof, alendronate sodium, etidronate disodium, pamidronate disodium, or an activator of parathyroid hormone (PTH) release.

As used herein, the term growth factor refers to any agent that stimulates cellular proliferation and/or differentiation. Growth factors include but are not limited to fibroblast growth factor (FGF), platelet-derived growth factor (PDGF), insulin-like growth factors (IGF) I and II, TGF-β, TGF-α, bone morphogenetic protein (BMP) (e.g., BMP-2, BMP-3, BMP-4, BMP-6, or BMP-7), hedgehog proteins, growth differentiation factors, hematopoietic colony-stimulating factors (CSF), vascular endothelium growth factor (VEGF), osteoid-inducing factor (OIF), angiogenins, endothelins, hepatocyte growth factor, keratinocyte growth factor, ADMP-1, interleukins (IL) (e.g., IL-3 and IL-6), epithelial growth factors, dexamethasone, leptin, sortilin, transglutaminase, prostaglandin E, 1,25-dihydroxyvitamin D3, ascorbic acid, pro-collagen, glycerol phosphate, TAK-778, statins, growth hormone, steel factor (SF), activin A (ACT), retinoic acid (RA), epidermal growth factor (EGF), hematopoietic growth factors, peptide growth factors, erythropoietin, tumor necrosis factors (TNF), interferons (IFN), heparin binding growth factor (HBGF), nerve growth factor (NGF) and muscle morphogenic factor (MMP).

In certain embodiments, particles further comprise whitening (coloring) agents, including but not limited to: peroxide, citroxain, titanium dioxide or sodium hexametaphosphate; abrasive agents, including but not limited to: silica, alumina, hydrated silica, dicalcium phosphate, salt, pumice, kaolin, bentonite, calcium carbonate (chalk), sodium bicarbonate (baking soda), or calcium pyrophosphate; active ingredients, including but not limited to: fluoride (sodium monofluorophosphate, stannous fluoride, or sodium fluoride), or xylitol (which reduces decay levels and enhances remineralization); antibacterial agents, including but not limited to: Triclosan, sanguinaria extract, baking soda, zinc citrate trihydrate, polyphenols, stannous fluoride, or essential oils; tartar control agents, including but not limited to: tetrasodium pyrophosphate, Gantrez 5-70, or sodium tri-polyphosphate; enzymes to improve antibacterial properties of saliva, including but not limited to; glucose oxidase, lactoperoxidase, or lysozyme; desensitizing agents, including but not limited to: potassium nitrate, strontium chloride, or sodium citrate; detergents, including but not limited to: sodium lauryl sulfate (SLS), sodium lauroyl sarcosinate, sodium N-lauryl sarcosinate, dioctyl sodium sulfosuccinate, sodium stearyl fumarate, sodium stearyl lactate, or sodium lauryl sulfoacetate; flavoring agents, including but not limited to: peppermint, spearmint, cinnamon, wintergreen, and menthol, or fennel; humectants, (which retain water and help maintain a consistent paste-like quality), including but not limited to: sorbitol, pentatol, glycerol, glycerin, propylene glycol, polyethylene glycol, water, xylitol, PEG 8 (polyoxyethylene glycol esters), or PPG (polyoxyethylene ethers); thickeners, including but not limited to: carrageenan, cellulose gum, xanthan gum, gum arabic, sodium carboxymethyl cellulose (CMC), cellulose ethers, sodium alginate, carbopols, silica thickeners, sodium aluminum silicates, or clays; preservatives, including but not limited to: sodium benzoate, methyl paraben, or ethyl paraben; sweeteners, including but not limited to: calcium or sodium saccharin, or aspartame (Nutrasweet); or other components, including but not limited to: stabilized chlorine dioxide, mellaleuca, neem, or CPP-ACP.

Any of the foregoing compositions may optionally further comprise at least one additional component, such as dental porcelain, dental amalgam, gypsum plaster impression material, gutta percha root canal filling, vulcanite, silicate cement, zinc phosphate cement, cobalt chromium base alloy, acrylic resin, alginate impression material, bis-GMA and composite resins, polysulphide impression material, zinc polycarboxylate cement, glass ionomer cement, dentine resin adhesive, amalgam, galloy, ceramic based cement, or bone cement.

In some embodiments the particles may comprise an agent, factor, or drug to promote the production of bone through mechanisms including promotion of osteogenic differentiation, collagen production, mineralization, osteogenic cell recruitment, or osteoclast suppression. In some embodiments, agents, factors, or drugs are incorporated within the particles through diffusion into pores within the particles.

In certain embodiments the particles may comprise cytokines and growth factors that may be exploited to promote bone growth. In other embodiments, the particles may contain a factor for promoting the formation of cartilage such as TGF-β. The particles may also contain a mechanism for promoting particle resorption leading to increased local calcium concentrations within the bone compartment. For example, in some embodiments, receptor activator of NFκ-B ligand (Rank-L), parathyroid hormone, macrophage colony stimulating factor (M-CSF), transforming growth factor-β1 (TOF-β1), or a corticosteroid such as dexamethasone could be used for this purpose. In other embodiments, any combination of the foregoing factors may be employed. In some embodiments the resorption factor may be incorporated onto the surface of the particles whereas in other embodiments the resorption factor may be incorporated into the bulk of the particle.

The particles may further contain agents to chelate minerals from blood within the bone compartment. In some embodiments, these agents are based on EDTA. In other embodiments the agent may be a poly(bisphosphonate) or poly(phosphate). In some embodiments, the particles may contain biological or non biological entities that nucleate calcium and/or phosphate. In certain embodiments, such entities may contain acid functionality such as aspartic acid. In other embodiments, nucleating agents may include osteopontin or bone sialoprotein.

Thus, the invention includes methods for delivering particles to a desired organ, tissue or cell of a subject. As used herein, the term “subject” refers to a human or non-human mammal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments the subject is a patient. As used herein, a “patient” refers to a subject who is under the care of a physician, dentist, or other health care worker, including someone who has consulted with, received advice from or received a prescription or other recommendation from a physician or other health care worker. A patient is typically a subject having or at risk of having a dental or bone disorder.

Aspects of the invention relate to methods for targeting a particle to a tooth. The method may be performed by contacting a tooth with a composition composed of a particle linked to a binding agent that interacts with a tooth molecule or a molecule on the surface of a tooth, such as a hydroxyapatite binding agent, in order to bind the particle to the tooth. The particle could be composed of an active agent or of a carrier and an active agent. The active agent may be, for instance, an agent that will cause whitening of the teeth. Calcium based particles are capable of providing a white surface when attached to the teeth. In the instance when a calcium based particle is used, it may be combined with other particulate carriers such as PLGA and or other whitening components or even any of the biological active agents described above, such as anti-microbials. The methods may also be achieved by contacting an oral cavity of a subject with a calcium particle linked to a saliva or bacterial binding agent to bind the calcium particle to saliva or bacteria on a tooth surface.

These methods for contacting teeth with particles may involve a single step. For instance a subject may apply the composition to the teeth using a solution such as a rinse where the particles are exposed to the teeth for a brief period of time to allow attachment. For instance the user may rinse the teeth for a few seconds to minutes, ie 5 seconds, 10 seconds, 20 seconds, 30 seconds, 1 minute or more. The composition may also be applied in other forms, such as pastes or strips.

Other methods of the invention involve a multi-step process. In this system the surface of a tooth may be modified and then contacted with a composition of a particle that is bound to a binding agent. For instance a first composition, a biological surface modifying agent, may be applied to a tooth surface. It may be applied by any known method, such as using a mouth rinse, a paste, strips, or painting it on etc.

A biological surface modifying agent is an agent that binds to the surface of the tooth or a component positioned on the surface of the tooth (i.e. bacteria) and includes at least one molecule capable of being bound. The biological surface modifying agent provides a coating on the tooth surface that can be used to adhere the particle. The biological surface modifying agent and the particle may each be composed of or contain a member of a binding pair. For instance the biological surface modifying agent may include an avidin and the particle may include a biotin. The particle may be composed of calcium and the biological surface modifying agent may include a calcium binder. An advantage of using a biological surface modifying agent is that any particle can be used as long as the biological surface modifying agent includes a binding partner. The particle does not need to include a component that binds to the tooth.

It is also possible that the multi step method may actually be performed as a single step. For instance the two compositions may be mixed together first and then applied simultaneously.

By way of non-limiting example, when the particle is nanoparticulate calcium carbonate and the binding agent is a fatty acid such as stearic acid, the composition comprising nanoparticulate calcium carbonate and stearic acid can be applied directly to a hydrophobic biological surface such as a tooth surface, or a biological surface treated to be hydrophobic, for example, by pretreatment with stearic acid. In another embodiment, the particle in a composition with a polyanionic binding agent will adhere directly to a cationic surface. In one example, a composition comprising cationic nanoparticulate calcium carbonate and anionic polycarboxylic acid will adhere directly to a cationic tooth surface. In another example, a cationic linker such as a polycarboxylic acid can be used to bridge nanoparticulate calcium carbonate to a tooth surface. In another embodiment, the tooth surface can be modified to become more cationic by application of a polymeric cation, and subsequently a composition comprising an anionic binding agent and any particle can be applied. By taking into account the surface charge (or lack thereof) on the biological surface; the surface charge (or lack thereof) on the particle, and intermediate components such as a binding agent, linker, and biological surface modifying agent, the appropriate conditions can be achieved to practice the invention.

Methods of the invention that relate to the binding of a particle to a tooth have many applications. For superficial applications or variable durations, applications include but are not limited to: tooth whitening, prevention of subsequent tooth staining events by providing a protective layer or by serving as a sink for staining agents including tobacco and coffee, inhibition of bacterial adhesion and growth on the tooth surface, prevention of plaque formation, prevention of gum recession by chelating calcium as plaque becomes calcified, and reduction of sensitivity to painful stimuli, such as heat and cold, for users with sensitive teeth. Longer term embedded applications for the binding of a particle to a tooth include but are not limited to: teeth whitening, anti-bacterial agents for use during invasive procedures (root canals etc.), prevention of the appearance of tooth death after root canals or operations within or beneath the gums, cavity filling agents, and increasing tooth strength especially with natural aging.

In some embodiments delivery of a particle to the oral cavity may further comprise a method for treating peridontal disease and/or gingivitis. As used herein peridontal disease or periodontitis refers to a variety of inflammatory diseases that affect the tissues that surround and support the teeth. Gingivitis as used herein, refers to an inflammation within the gums, or around a tooth. Since these conditions are frequently caused by bacterial plaque that accumulates in the spaces between the gums and the teeth, and in tartar that forms on the teeth, delivery of a particle containing an agent such as an anti-bacterial agent to the area surrounding a tooth may be effective in the treatment of these conditions.

By using particles with particular morphologies or chemistries, in one embodiment, the particles could serve to mask underlying stains and prevent bacterial adhesion to teeth thus reducing the incidence of carries and halitosis. For prevention of bacterial adhesion, particles may be modified with super positively charged, antibacterial groups or other antibacterial agents. Alternatively the particles may be coated with polymers using layer-by-layer deposition or direct embedding within polymers. The number of layers used via the layer-by-layer deposition could be used to alter the degree of whitening of the tooth and stability of the coatings on the tooth. In another embodiment, these particles can be applied to aid in the remineralization of enamel as desired, or as appropriate for the specific application.

In other embodiments of the invention, the aforementioned principles can be applied to any biological surface or artificial biological surface of a human or other animal body, such as but not limited to tooth, dental implant, bone, tissue, medical implant, skin, mucosa or hair. Furthermore, in addition to coloration of the surface, other properties may be provided or imparted to any such biological surface, such as but not limited to color, sheen, texture, mineral content, mineral composition, taste, plaque content, odor, odor production, and any combination thereof. In some embodiments, modifications of biological surface sheen may be achieved by coating particles of the invention with a lac resin such as shellac or pharmaceutical glaze. In other embodiments, a lac resin can be incorporated into a composition by copolymerization.

Aspects of the invention relate to methods for applying a particle or a composition to a tooth. In some embodiments, the particle may remain on the tooth during eating and other activity, and can be removed at the wearer's discretion, to leave the tooth as it appeared before the application of the particle. In other embodiments the method comprises applying a particle to a tooth and selectively removing the particle from the tooth. In other embodiments the method for applying a particle to a tooth can be repeated by the wearer, to change the appearance of a tooth, on a regular basis if the wearer desires, without having damaging effects on the tooth enamel. Other embodiments of the invention provide a method which includes a color matching system, wherein the user mixes components to colorize the composition containing a treatment agent to be applied to the tooth to the desired hue or color, which can be a tooth color or a non-tooth color. In other embodiments the components are already mixed to achieve the one or more desired properties.

Other embodiments of the invention involve repeating application and removal of the compound containing a treatment agent on a tooth for different colors as the user determines and selects. In another embodiment, thermosensitive polymer coatings such as carageenan or polyNiPAAm may be used to facilitate easy removal.

In another embodiment, a tooth covering composition is provided which can be selectively colorized, applied and removed by a user.

In some embodiments of the invention, the particles may be used to bond to stain-causing components of drinks and foods and thus prevent their adherence to teeth. These particles could be used as an additive for toothpaste or mouthwash, included in food products, taken as a supplement before or after eating to inhibit the staining process, or used as a supplement to bleach (included in the bleach product or as a second material that can be intermixed) to reduce the likelihood of agents staining teeth during the period of increased sensitivity to discoloring agents.

In some embodiments, the biological surface may be modified in order to promote the association of a particle with the biological surface. In some embodiments modification of the biological surface may comprise removing the protein layer on the teeth. This can be achieved, for example, by applying dilute aqueous acetic acid or phosphoric acid to the tooth surface. As used herein a dilute solution refers to a solution containing a relatively small quantity of solute as compared with the amount of solvent In some embodiments, a dilute acid solution may be a less than 1%, less than 5%, less than 10%, or less than 20% solution. In certain embodiments, the tooth surface may be brushed to remove the protein layer. In some embodiments, the administration of the adherent particles to a tooth may require an initial brushing, rinsing and/or drying step to remove bacteria, debris, and/or moisture that coats the tooth surface.

Aspects of the invention relate to methods and compositions for applying particles to teeth. In some embodiments, the composition or any component thereof is provided in a form including but not limited to: dentifrices including mouthwashes, mouthrinses, toothpastes, tooth powders, tooth hardeners, antiplaque compositions, dental creams, dental flosses, liquids, gels, chewing gums, including center-filled gums, and confectionaries including mints and lozenges. As used herein the term dentifrice refers to a paste, powder, liquid, or other preparation for cleaning the teeth. In certain embodiments, the compositions of the invention are in the form of chewing gums. In other embodiments, liquid formulations such as mouthrinses and liquids can comprise the particulates in the form of a colloidal suspension. As used herein a liquid formulation refers to a substance that flows readily and is neither solid not gaseous A colloidal suspension as used herein refers to a system in which finely divided particles are dispersed within a continuous medium. Colloidal suspensions are useful for treating halitosis or reducing plaque formation in animals. In some embodiments the compositions of the invention may be added to drinking water.

Particles and compositions containing particles may be used in the form of a gel, paste, gum or oral rinse. The particles may also be incorporated into a resin that can be polymerized on the tooth surface. Similarly particles in these or other. formulations can be used for direct application within teeth.

In some embodiments, methods for applying a particle or composition to a biological surface may be carried out by means of an applicator. Non-limiting examples include a syringe for depositing a composition at a particular location or along a surface, or, in the example of application to teeth, a semicircular trough that can be filled with a composition then held along the upper or lower row of teeth for a desired period of time, contacting all or a subset of the teeth with the composition.

Among the foregoing embodiments, depending on the extent to which the alteration of the biological surface property is desired, the steps of the various embodiments can be repeated to apply multiple layers of the particle or composition to the biological surface. For example, to mask the color of stained teeth or to make teeth appear whiter, multiple applications of the compositions herein may be undertaken during a treatment period. In other embodiments, the method may be carried out on a regular basis, for example, daily, weekly or monthly, for the duration of having the desired biological properties modified. In certain embodiments, the property modification is temporary. In some embodiments, temporary refers to a duration of time from about 30 minutes to about 2 hours, about 2 hours to about 6 hours, about 6 hours to about 12 hours, about 24 hours to about one week, or about 1 week to about 6 weeks. In other embodiments, temporary refers to the period between consuming meals.

In certain embodiments of the invention related to tooth whitening, multiple particles or compositions could be mixed either at the point-of-care or at synthesis to produce desired shades of white (or other colors if so desired). A variety of base colors may be fabricated through providing various mixtures of particles having different shades of white (or other colors). The patient could be presented with a variety of products that present various shades of white to choose from. Alternatively, similar to the paint industry, patients may tailor a composition to a desired color. In other embodiments, these particles may be used in other areas of cosmetics to alter skin color (pigment enhancer), cover-up red-eye, cover-up moles or blemishes. This technology may also be applicable to altering color on surfaces for other household or industrial applications.

The particles may also be incorporated within other materials that can be used to coat surfaces, such as, but not limited to hydrogels.

The compositions of the invention may also be used for delivery of components to bone. For instance, the particles may be used to localize calcium to bone, where it can be incorporated into bone as it forms and/or is remodeled. In some embodiments, calcium and/or carbonate from the particles is incorporated into newly formed bone, resulting in increased local bone mass. In other embodiments, phosphorous or other bone containing inorganic minerals from the particle is incorporated into the newly formed bone thus increasing bone mass.

The methods may be performed, for instance, by administering a calcium particle that is linked to a modifying ligand and/or a hydroxyapatite binding agent to a subject to deliver the calcium particle to the bone. The methods may be useful simply for preventative purposes, such as the routine delivery of calcium to the bone or for therapeutic or diagnostic purposes. For instance the particles may be used to carry a diagnostic agent to the bone. Alternatively the particles may be used therapeutically in the treatment of bone disorders.

The particles can be delivered to a subject and reach their desired targets through a variety of mechanisms. The method of administration could include but is not limited to oral, intravenous injection, subcutaneous injection, parenteral, rectal, sublingual, transdermal, nasal, inhalable, and ocular administration. In some embodiments the particles may reach the bone compartment through traveling through the blood stream. In some embodiments the particles may be delivered as a suspension, solution, solid, injected directly into a vessel, ingested as part of a drink, pill, gum, gel, inhaled, or transdermally administered. In other embodiments, the particles may be injected directly into affected bones. In some embodiments, a combination of different particle types may be administered to increase bone mass via more than one mechanism (for example, particles that stimulate bone growth combined with particles that increase mineralization of the produced matrix).

Delivery of calcium to bone has applications for a variety of disorders that weaken bone strength, including but not limited to bone cancer, osteoporosis or other low bone mass diagnoses, osteopetrosis, avascular necrosis, fibrous dysplasia, osteogenesis imperfecta, osteomyelitis, Paget's disease of the bone, and primary hyperparathyroidism. Osteoporosis is a disease of bone in which the bone mineral density (BMD) is reduced, microarchitecture is disrupted, and the amount and variety of non-collagenous proteins in bone is altered. Calcium deficiency increases the risk of osteoporosis. Thus, an effective means of delivering calcium particles to bone may provide a treatment strategy for osteoporosis. As well, the particles described in the instant invention may allow for delivery of particles containing drugs for treatment of osteoporosis to osteoporotic bone, assisting in treatment of this disorder. Other low bone mass diagnoses besides osteoporosis which have a similar etiology to osteoporosis may similarly be treated by the particles described in the instant invention.

The most common form of malignant bone cancer is osteosarcoma wherein neoplastic cells present osteoblastic differentiation and form tumoral bone. Osteopetrosis refers to a rare inherited disorder whereby the bones harden, becoming denser. People with osteopetrosis have excess bone formation but tend to have bones that are more brittle than normal. Avascular necrosis is a disease resulting from the temporary or permanent loss of blood supply to the bones. Without blood, the bone tissue dies and causes the bone to collapse. Fibrous dysplasia is a disease that causes growths or lesions in one or more bones of the human body. These lesions are tumor-like growths that consist of replacement of the medullary bone with fibrous tissue, causing the expansion and weakening of the areas of bone involved. Osteogenesis imperfecta is a group of genetic bone disorders. It is one of the brittle bone diseases and is associated with a decreased amount of collagen. Osteomyelitis is an infection of bone, usually caused by pyogenic bacteria or mycobacteria. Paget's disease of the bone, also known as osteitis deformans is a rarefying osteitis resulting in weakened, deformed bones of increased mass. Hyperparathyroidism refers to excessive activity of the parathyroid glands and can lead to alteration in function of bone cells, and tends to occur when the serum calcium falls below normal, as in chronic renal disease. For the foregoing examples of disease conditions, one can envision the advantage of a treatment system which can deliver particles of calcium, a core constituent of bone, and which can also deliver agents, factors or drugs, directly to bone, as is described in the instant invention.

In some embodiments a particle may be modified through the introduction of a modifying ligand. A modifying ligand, as used herein is a molecule that alters the properties of a particle. Properties include, for instance, the degradation rate of the particle and the ability of the particles to be recognized by bodily components such as the immune system or other cells. An example of a modifying ligand is a stealth ligand. A stealth ligand aids in increasing circulation time by resisting uptake by cells that would absorb and metabolize the particles. The modifying ligand can also be an effector ligand that helps to facilitate bone growth, mineralization, and/or bone turnover. Particles can have one or more modifying ligands. In instances with multiple ligands attached, common or distinct linkers can be used for each ligand. In some embodiments a linker may be used to attach the modifying ligand to a particle. In certain embodiments the linker may comprise poly(ethylene glycol), hyaluronic acid, dextran, chitosan, or poly(ethylene oxide). In some embodiments the linker may serve as a stealth ligand where it achieves the same function while enabling linkage to a different modifying linker or when the linker is selected from the group that composes the stealth ligands, and has no other ligand attached to it.

Particles may reach the bone compartment through the aid of binding agents. In some embodiments uptake to the bone compartment may be either non-specific or augmented through the binding agent linked to the particle. Given the potential for non specific uptake in non bony tissues such as kidney, liver, and lungs, in some embodiments, modifying ligands such as hydroxides, attached to the particles, may act to prevent nucleation leading to kidney stones.

In some embodiments, the circulation time of a particle may be increased through incorporation of a stealth ligand into the surface of the particle. In other embodiments the stealth ligand is incorporated into the bulk of the particle and thus continuously expressed during dissolution of the particle. In certain embodiments, the stealth ligand may comprise poly(ethylene glycol). In other embodiments the stealth ligand may comprise hyaluronic acid, dextran, chitosan, or poly(ethylene oxide). Incorporation of a stealth ligand may enhance clinical success by avoiding potential calcification in tissues such as liver and spleen that could otherwise occur following prolonged systemic administration of particles containing calcium.

The particles can be included into delivery vehicles to control the release of particles into the bloodstream and/or bone. In some embodiments, the particle may contain a mechanism to control the delivery of calcium within the bone compartment. In certain embodiments, this mechanism consists of combining the calcium salt with a degradable polymer such as poly hydroxy acids such as poly(lactide-co-glycolide) (PLGA). In other embodiments this polymer may comprise dextran, hyaluronic acid, poly(citrate), poly(glycerol sebacate), chitosan, elastin, or poly(carbonate).

As used herein, the term treat, treated, or treating when used with respect to a disorder such as osteoporosis refers to a prophylactic treatment which increases the resistance of a subject to development of the disease or, in other words, decreases the likelihood that the subject will develop the disease as well as a treatment after the subject has developed the disease in order to fight the disease or prevent the disease from becoming worse.

The term “effective amount” of a particle of the invention refers to the amount necessary or sufficient to realize a desired biologic effect. For example, an effective amount of particles for treating osteoporosis is that amount sufficient to prevent an increase in bone loss in the subject or that amount necessary to decrease the amount of further damage to the bone that would otherwise occur in the absence of the particles. An effective amount of the particles for promoting teeth whitening, for instance, is that amount that increases the white color or shine or reduces stains. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular composition being administered, the size of the subject, or the severity of the disease or condition. One of ordinary skill in the art can empirically determine the effective amount of a particular composition of the invention without necessitating undue experimentation.

The particles of the invention may be delivered to the subject on an as needed or desired basis. For instance a subject may self administer the particles as desired in order to whiten teeth or a dentist may administer the particles during a routine visit. Additionally a physician or other health care worker may select a delivery schedule. In other embodiments of the invention, the particles are administered on a routine schedule. A “routine schedule” as used herein, refers to a predetermined designated period of time. The routine schedule may encompass periods of time which are identical or which differ in length, as long as the schedule is predetermined. For instance, the routine schedule may involve administration of the composition on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the composition on a daily basis for the first week, followed by a monthly basis for several months, and then every three months after that. Any particular combination would be covered by the routine schedule as long as it is determined ahead of time that the appropriate schedule involves administration on a certain day.

The particles may be administered alone or in any appropriate pharmaceutical carrier, such as a liquid, for example saline, or a powder, for administration in vivo. They can also be co-delivered with larger carrier particle or within administration devices. The particles may be formulated. The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the particles can be administered to a subject by any mode that delivers the particles to the desired surface, e.g., teeth, bone. Administering the pharmaceutical composition of the present invention may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

It is well known to those skilled in the art that particles may be administered to patients using a full range of routes of administration. As an example, particles may be blended with direct compression or wet compression tableting excipients using standard formulation methods. The resulting granulated masses may then be compressed in molds or dies to form tablets and subsequently administered via the oral route of administration. Alternately particle granulates may be extruded, spheronized and administered orally as the contents of capsules and caplets. Tablets, capsules and caplets may be film coated to alter dissolution of the delivery system (enteric coating) or target delivery of the particle to different regions of the gastrointestinal tract Additionally, particles may be orally administered as suspensions in aqueous fluids or sugar solutions (syrups) or hydroalcoholic solutions (elixirs) or oils. The particles may also be administered directly by the oral route without any further processing.

The particles of the invention may be systemically administered in combination with a pharmaceutically acceptable vehicle such as an inert diluent or an assimilable edible carrier. They may be enclosed in hard or soft shell gelatin capsules or compressed into tablets. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound, ie calcium. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed.

To ensure full gastric resistance a coating impermeable to at least pH 5.0 is helpful. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The particles of the invention may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In some embodiments the compositions of the invention are not encapsulated or formulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For topical administration, the particles of the invention will generally be administered as compositions or formulations, in combination with a dermatologically acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The compositions of the inventions may include a physiologically or pharmaceutically acceptable carrier, excipient, or stabilizer mixed with the particles. The term “pharmaceutically acceptable” means a non-toxic material that does not interfere with the effectiveness of the biological activity of the active ingredients. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency. A pharmaceutical preparation is a composition suitable for administration to a subject. Such preparations are usually sterile and prepared according to GMP standards, particularly if they are to be used in human subjects. In general, a pharmaceutical composition or preparation comprises the particles, and optionally agents of the invention and a pharmaceutically-acceptable carrier, wherein the agents are generally provided in effective amounts.

Particles may also be suspended in non-viscous fluids and nebulized or atomized for administration of the dosage form to nasal membranes. Particles may also be delivered parenterally by either intravenous, subcutaneous, intramuscular, intrathecal, intravitreal or intradermal routes as sterile suspensions in isotonic fluids.

Finally, particles may be nebulized and delivered as dry powders in metered-dose inhalers for purposes of inhalation delivery. For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of for use in an inhaler or insufflator may be formulated containing the microparticle and optionally a suitable base such as lactose or starch. Those of skill in the art can readily determine the various parameters and conditions for producing aerosols without resort to undue experimentation. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Techniques for preparing aerosol delivery systems are well known to those of skill in the art. Generally, such systems should utilize components which will not significantly impair the biological properties of the agent in the nanoparticle or microparticle (see, for example, Sciarra and Cutie, “Aerosols,” in Remington's Pharmaceutical Sciences, 18th edition, 1990, pp. 1694-1712; incorporated by reference).

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass..

Particles when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The present invention also provides any of the above-mentioned compositions in kits. In some embodiments a kit for binding a particle or composition to a tooth comprises a container housing a biological surface modifying agent, a second container housing a calcium particle linked to a binding agent, and instructions for applying the biological surface modifying agent and the calcium particle to a tooth. In certain embodiments, instructions are provided for blending the two agents to a desired intermediate property. In some embodiments, the blending is to provide a desired final color imparted to the biological surface, such as the tooth surface.

In some embodiments, a kit further includes an applicator to apply one or more the components to the biological surface, for example, a syringe-type applicator for depositing material on the biological surface or, for example, an applicator in the shape of a semicircular trough for use in applying material to the teeth.

The kits described in the invention optionally include instructions for use of the composition for application to teeth or for the treatment of a condition. That is, the kit can include a description of use of the composition for participation in any biological or chemical mechanism disclosed herein. The kits can further include a description of activity of the condition in treating the pathology, as opposed to the symptoms of the condition. That is, the kit can include a description of use of the compositions as discussed herein. The kit also can include instructions for use of a combination of two or more compositions of the invention, or instruction for use of a combination of a composition of the invention and one or more other compounds indicated for modification of a surface such as a tooth or treatment of a condition such as a bone disorder. Instructions also may be provided for administering the composition by any suitable technique as previously described.

The kits described herein may also contain one or more containers, which may contain the composition and other ingredients as previously described. The kits also may contain instructions for mixing, diluting, and/or administering or applying the compositions of the invention in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

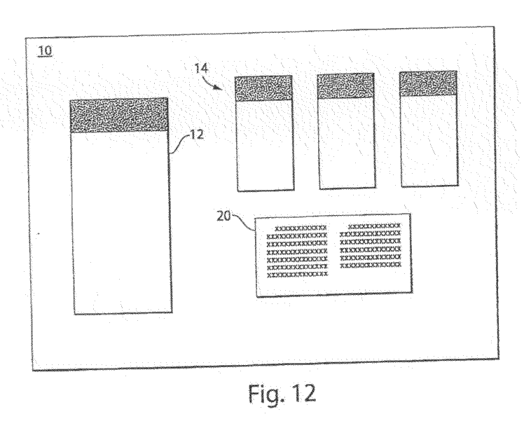

An example of a kit useful according to the invention is shown in FIG. 12. The kit (10) shown in FIG. 12 includes a set of containers for housing particles (12) and other compounds (14) as well as instructions (20).

### EXAMPLES

### Example 1

### Layer by Layer (LBL) Formulation of CaCO₃-Alginate-PLL Particles

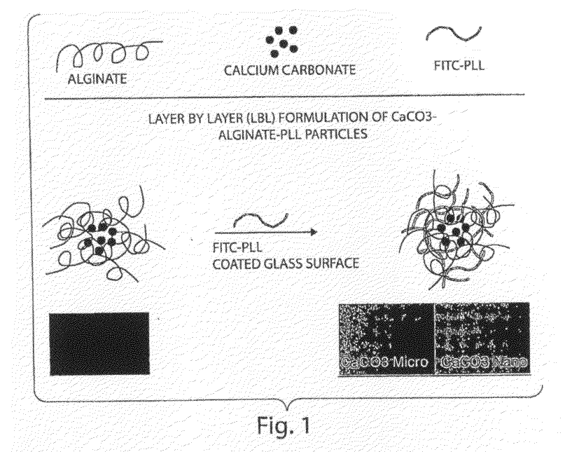

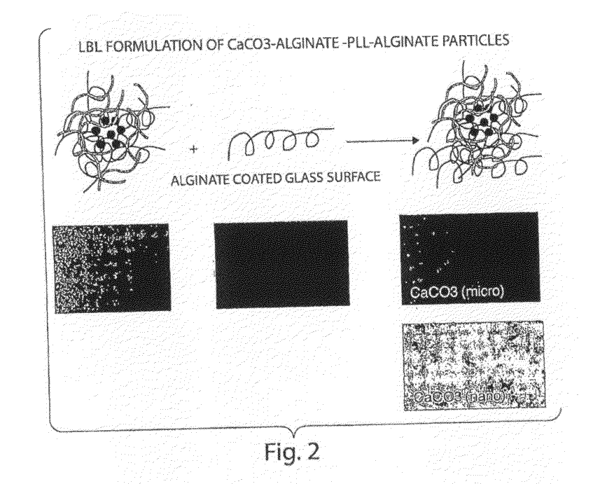

In this experiment, CaCO₃ particles (microparticles and nanoparticles) were suspended in double-distilled (dd) H2O at (30 v/v %) then mixed with 1% sodium alginate solution. The resulting CaCO₃-alginate mixture was collected by centrifugation at 10008 for 5 minutes, and washed 3× by resuspending the particles in ddH20. The particles were coated onto a glass surface coated with 0.01% of Poly-L-Lysine-FITC, then imaged under a fluorescent microscope. All images were taken under the same gain and exposure setting. The layer by layer (LBL) formulation of CaCO₃-alginate-PLL particles on a glass surface is shown in FIG. 1. Layer by layer (LBL) formulation of CaCO₃-alginate-PLL-alginate particles on an alginate-coated surface is shown in FIG. 2.

### Example 2

### Layer by Layer (LBL) Formulation of Alginate and PLL

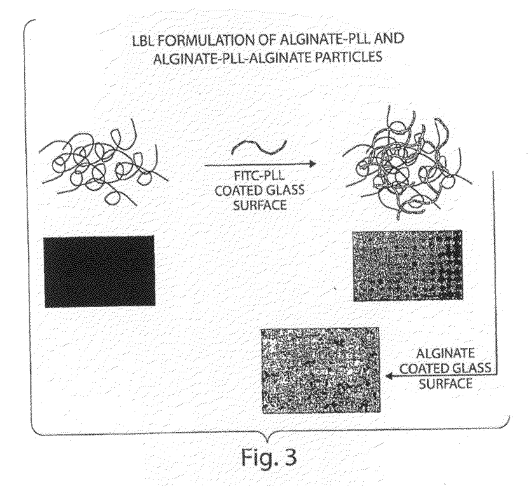

In this experiment, Alginate-PLL particles were formed by placing 1% sodium alginate solution on a glass surface coated with 0.01% PLL-FITC. Alginate-PLL-alginate particles were formed by placing a suspension of alginate-CaCO₃ on a glass surface coated with 2% sodium alginate. Both alginate-PLL and alginate-PLL-alginate particles were washed 3× in ddH20 before being placed onto the pretreated glass surface. The layer-by-layer (LBL) formulation of alginate and PLL on an alginate-coated glass surface is depicted in FIG. 3.

### Example 3

### Separation of Unbound PLL-FITC from CaCO₃-Alginate-PLL Particles

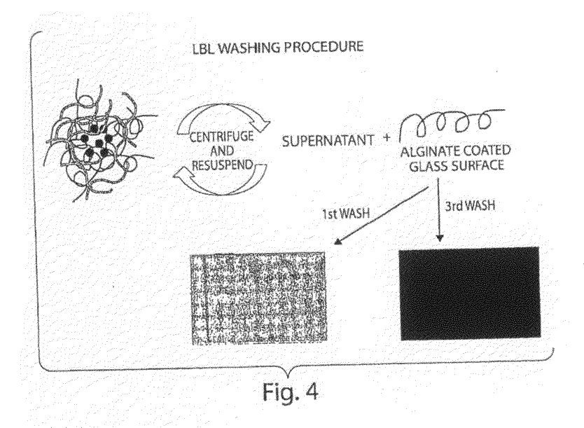

CaCO₃-alginate particles were mixed with PLL-FITC solution according to the procedure described in Example 1. The particles were then collected by centrifugation and the supernatant after each wash was placed on a glass surface pretreated with 1% alginate. FIG. 4 depicts the separation of the unbound PLL-FITC from the solution containing CaCO₃-alginate-PLL particles.

### Example 4

### Preparation of CaCO₃-CMC-PLL and CaCO₃-PSS-PLL Particles

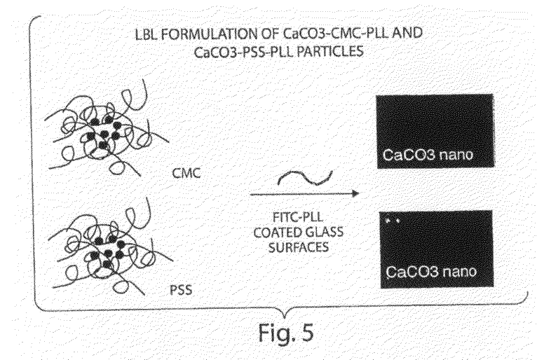

CaCO₃ particles were suspended in ddH20 at (30 v/v %) then mixed with 2% CMC (carboxymethylcellulose) or PSS (polystyrene sulfonic acid) solution. The resulting CaCO₃CMC or CaCO₃-PSS mixtures were washed and collected according to the procedure described in Example 1. The particles were coated onto a glass surface coated with 0.01% Poly-LLysine-FITC, then imaged under a fluorescent microscope. FIG. 5 shows the binding of CaCO₃-CMC-PLL and CaCO₃-PSS-PLL particles to glass surfaces coated with 0.01% Poly-L-Lysine-FITC.

### Example 5

### Binding of Bisphosphonate Functionalized Biodegradable Nanoparticles to CaCO₃ Particles

50 mg of PLGA-PEG nanoparticles were made by precipitating PLGA-PEG diblock copolymer in sterile water. The biodegradable nanoparticles were composed of a diblock copolymer poly(D,L-lactide-co-glycolide)-co-poly(ethylene glycol), (PLGA-PEG). Bisphosphonate was conjugated to the carboxyl terminal of PEG by EDC/NHS chemistry. To conjugate Bisphosphonate to the surface of nanoparticle, the carboxyl groups on the PEG termini were activated by EDC and NHS (100 molar excess of PLGA-PEG), followed by mixing with bisphosphonates at a molar ratio of 1:10 (PLGA-PEG:Bis). The particle size was analyzed using Phase Analysis Light Scattering detector (Brookehaven).

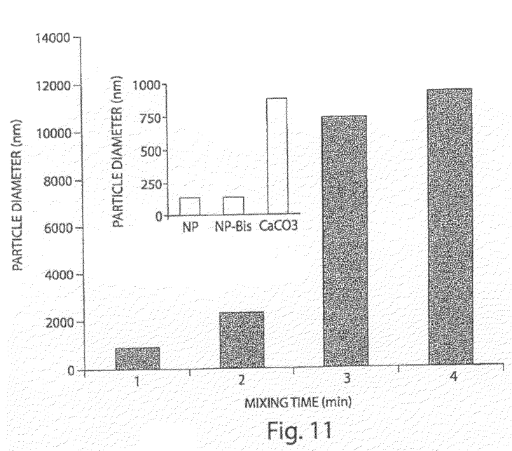

By adding trace amounts of bisphophonate-PEG-PLGA nanoparticles (average size 135 nm) to a solution of CaCO₃ (average size 800 nm), CaCO₃ particles underwent significant aggregation within 3 minutes. N=3. FIG. 11 is a graph demonstrating aggregation of CaCO₃ particles in a solution containing bisphosphonate-poly(D,L-lactide-co-glycolide)-co-poly(ethylene glycol) (PLGA-PEG) nanoparticles. Control groups consisting of solutions which contained bisphosphonate with the calcium carbonate alone, plain PLGA particles with calcium carbonate in the presence of bisphosphonate, and EDC/NHS activated PLGA particles with the calcium carbonate, did not show any aggregation. This data supports the potential for us to first coat the tooth surface (exposed hydroxyapatite) with an agent that binds calcium to promote binding of calcium containing particles from solution. It also suggests that particles that are functionalized with a divalent cation targeting agent should attach to surfaces that contain divalent cations (i.e. hydroxyapatite). This use of PLGA-PEG nanoparticles also suggest that particles can be used as a drug delivery vehicle with the capability of delivering both hydrophilic and hydrophobic agents at a sustained rate. These data also demonstrate rapid binding between calcium containing particles and the bisphosphonate-functionalized nanoparticles, which demonstrates the development of commercializable products capable of rapid onset binding to teeth upon administration.

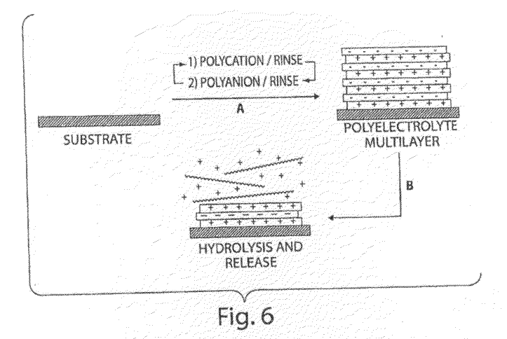

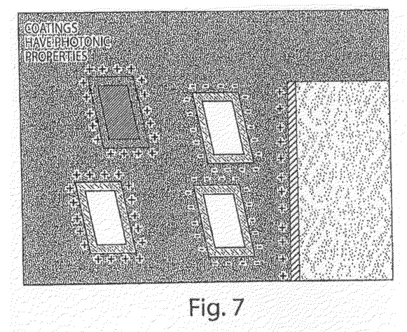

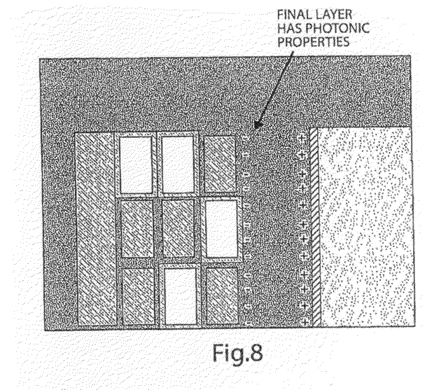

Several schematics of layering processes are shown in FIGS. 6-8. FIG. 6 shows a schematic of the layer-by-layer approach to providing altering agent on a biological surface. In this approach, cationic and anionic polymers can self-assemble onto teeth surface by electrostatic interaction. Therapeutic and/or cosmetic agents can be encapsulated in between the layers of oppositely charged layers. Controlled release of these agents can be achieved when the polymer complex disassemble in a layer by layer fashion. FIG. 7 shows the deposition of compositions of alternating charges containing an altering agent. This process can co-encapsulate at least two different therapeutic/cosmetic agents, and subsequently release them in different kinetics. FIG. 8 shows another example in which layers are built up prior to application to the surface. This approach allows targeted delivery of nanoparticles containing therapeutic/cosmetic agents by coating the teeth surface with a layer of targeting ligands.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A composition comprising a calcium particle linked to a hydroxyapatite binding agent.

2-4. (canceled)

5. The composition of claim 1 wherein the calcium particle is calcium carbonate.

6. (canceled)

7. The composition of claim 1 wherein the diameter of the calcium particle is about 5 nm to about 500 nm.

8-9. (canceled)

10. The composition of claim 1 wherein the particle comprises an aggregate of particles.

11-17. (canceled)

18. The composition of claim 1 wherein the composition comprises a dentifrice including a mouthwash, a mouthrinse, a toothpaste, a tooth powder, a tooth hardener, an antiplaque composition, a dental cream, a dental floss, a liquid, a gel, a chewing gum, including a center-filled gum, a confectionary, including mints, lozenges.

19-21. (canceled)

22. The composition of claim 1 wherein the calcium particle is further linked to a modifying ligand and a hydroxyapatite binding agent.

23. (canceled)

24. The composition of claim 22 wherein the modifying ligand comprises a stealth ligand.

25-26. (canceled)

27. A composition comprising a solution of particles linked to a hydroxyapatite binding agent, wherein the particle is not incorporated into a liposome.

28-30. (canceled)

31. The composition of claim 27 further comprising a biodegradable polymer.

32. The composition of claim 27 wherein the particle consists essentially of water insoluble components.

33. A method comprising
contacting a tooth of a subject with a composition comprising a calcium particle linked to a hydroxyapatite binding agent, to bind the calcium particle to the tooth.

34. The method of claim 33 wherein the binding of the particle to the tooth promotes whitening of the tooth.

35-37. (canceled)

38. The method of claim 33 wherein the method further comprises a method for treating a disease selected from the group consisting of peridontal disease and gingivitis.

39. (canceled)

40. A method comprising
contacting a tooth of a subject with a particle linked to a hydroxyapatite binding agent, wherein the particle is not incorporated into a liposome, to bind the particle to the tooth.

41-46. (canceled)

47. A method comprising
(a) applying to the tooth of a subject a biological surface modifying agent, and
(b) contacting the biological surface modifying agent with a composition comprising a calcium particle linked to a binding agent wherein the binding agent binds to the biological surface modifying agent on the surface of the tooth to bind the particle to the tooth.

48-56. (canceled)

57. A method comprising
contacting an oral cavity of a subject with a calcium particle linked to a saliva or bacterial binding agent to bind the calcium particle to saliva or bacteria on a tooth surface.

58-71. (canceled)

72. A method for delivering calcium particles to bone comprising administering to a subject a calcium particle linked to both a modifying ligand and a hydroxyapatite binding agent, to deliver the calcium particle to the bone.

73. The method of claim 72 wherein the modifying ligand comprises a stealth ligand.

74. (canceled)

75. The method of claim 72 wherein the particle further comprises an agent, factor or drug.

76-102. (canceled)

103. A method for treating osteoporosis comprising administering to a subject having or at risk of having osteoporosis an effective amount of a calcium particle linked to both a modifying ligand and a hydroxyapatite binding agent to treat osteoporosis in the subject.

104-132. (canceled)

133. A composition comprising a water insoluble particle containing an agent that promotes targeting of the particle to a calcium containing substrate.

134. The composition of 133 where the calcium containing substrate is hydroxyapatite.

135. The composition of claim 133 wherein the particle further comprises a drug.

136. The composition of claim 135 wherein the drug is an anti-neoplastic agent.

137. The composition of claim 133 wherein the particle further comprises a bone targeting agent which targets a bone marrow specific membrane surface receptor.

138. A composition comprising a solution of particles linked to a mineral binding agent, wherein the particle is not incorporated into a liposome.

139. A method comprising
contacting a tooth of a subject with a composition comprising a calcium particle linked to a mineral binding agent, to bind the calcium particle to the tooth.

140. A method comprising
(a) applying to a tissue a biological surface modifying agent, and
(b) contacting the biological surface modifying agent with a composition comprising a calcium particle linked to a binding agent wherein the binding agent binds to the biological surface modifying agent on the surface of the tissue to bind the particle to the tissue

141. A method for delivering a drug to bone, comprising
contacting a bone tissue with a composition comprising a water insoluble particle containing an agent that promotes targeting of the particle to hydroxyapatite, wherein the particle further comprises a drug.
